(19) United States
(12) Patent Application Publication
(10) Pub. No.: US 2009/0170937 A1
(43) Pub. Date: Jul. 2, 2009

(54) Amino Acid Derivatives

(75) Inventor: Christopher Hobbs, London (GB)

Correspondence Address: BANNER & WITCOFF, LTD., 1100 13th STREET, N.W., SUITE 1200, WASHINGTON, DC, 20005-4051, US

(73) Assignees: Proximagen Ltd., London (GB); Hodgkin Building,King`s College London,Guy`s Compus, London SE1 1UL (GB)

(21) Appl. No.: 12/278,437

(22) PCT Filed: Jan. 23, 2007
(86) PCT No.: PCT/GB2007/000181

§ 371 Date: Aug. 6, 2008

(30) Foreign Application Priority Data
Feb. 11, 2006 (GB) 0602780.9

(51) Int. Cl. A61K 31/216; C07C 229/34; A61P 25/16; A61P 25/14

(52) U.S. Cl. 514/533; 560/40; 514/542

(57) ABSTRACT

Compounds of formula (I) have activity in alleviating the effects of impaired dopaminergic signaling, for example in the treatment of Parkinsons Disease:

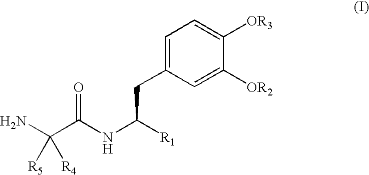

wherein: R₁ is a carboxyl, carboxyl ester, or carboxamide group; R₂ and R₃ are independently hydrogen, or a group —C(═O)R₆ or —C(═O)OR₆ wherein Re is C₁-C₆ alkyl, or a group —CH₂Q wherein Q is an optionally substituted monocyclic carbocyclic or heterocyclyl ring of 3 to 6 ring atoms; R₄ and R₅ are independently (a) the side chain of a natural amino acid, or (b) optionally substituted C₁-C₄ alkyl, C₂-C₄ alkenyl, or C₂-C₄ alkynyl, or (c) —CH₂XCH₃, —CH₂CH₂XCH₃, or —CH₂XCH₂CH₃, wherein X is —O—, S, or —NR₇ wherein R₇ is hydrogen, methyl or ethyl; or (d) —CH₂Q or CH₂OQ wherein Q is as defined in relation to R₆; or R₄ and R₅ taken together with the carbon atom to which they are attached form an optionally substituted cycloalkyl or heterocyclic ring of 3 to 8 ring atoms, optionally fused to a second, optionally substituted, carbocyclic or heterocyclic ring

The present invention relates to compounds which diminish the symptoms of dopamine deficiency.

Dopamine is a substance produced naturally by neurons in the basal ganglia of the brain that allows smooth, coordinated control of voluntary movement. Loss of, or impairment of, dopamine-producing neurons in the brain is implicated in Parkinson's disease and related parkinson-plus syndromes. These conditions respond to dopamine replacement therapy. Other conditions, for example, Restless Legs Syndrome (RLS) also respond to dopamine replacement therapy.

Parkinson's disease is a progressive neurodegenerative disorder that affects neuronal cells in the substantia nigra in the mid-brain. It is an age-related disorder of the central nervous system primarily attacking people over the age of 60. Approximately one out of every 500 people contract the illness and approximately one out of every 100 people over the age of 60 develop the illness. As indicated above, Parkinson's Disease is thought to be caused by a deficiency of dopamine. The common symptoms include tremor, stiffness (or rigidity) of muscles, slowness of movement (bradykinesia) and loss of balance (postural dysfunction). Parkinson's Disease is one of the most prevalent neurodegenerative illnesses. The natural history of the disease is progressive and from 10-15 years from onset of the disease becomes disabling in most patients.

Parkinson's disease is largely sporadic and referred to as idiopathic in nature. Forms of the illness due to vascular incidents and to toxin exposure also exist. Rare familial forms of the illness also exist.

Many treatments have been tried since James Parkinson first described the condition in 1817. Current therapy for Parkinson's disease is based on dopamine replacement therapy based on the use of the dopamine precursor levodopa (or L-dopa) or dopaminergic compounds. L-dopa is highly effective in reversing the motor symptoms of the illness but on chronic treatment and with disease progression, its effectiveness declines. The duration of drug response is reduced and unpredictable fluctuations in movement occur. Treatment is associated with therapy limiting side effects which include involuntary movements (dyskinesia) and psychosis.

RLS is a neurosensorimotor disorder with parestethesias, sleep disturbances and, in most cases, periodic limb movements of sleep (PLMS). Two forms of RLS appear to exist: the idiopathic and the uremic form. RLS is characterised by (1) a desire to move the legs, usually associated with paresthesias/dysesthesias, (2) motor restlessness, (3) worsening or exclusive presence of symptoms at rest (i.e. lying, sitting) with at least partial or temporary relief by activity, and (4) worsening of symptoms during the evening or night.

The present invention provides compounds which are active as dopaminergic compounds or as compounds which or as compounds which diminish the symptoms of dopamine deficiency.

According to the invention, there is provided a compound of formula (I) or a salt, hydrate or solvate thereof:

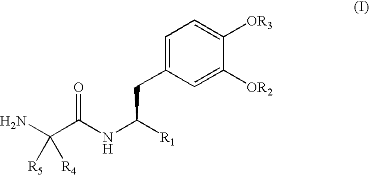

wherein:
R₁ is a carboxyl, carboxyl ester, or carboxamide group;
R₂ and R₃ are independently hydrogen, or a group —C(═O)R₆ or —C(═O)OR₆ wherein R₆ is C₁-C₆ alkyl, or a group —CH₂Q wherein Q is an optionally substituted monocyclic carbocyclic or heterocyclyl ring of 3 to 6 ring atoms;
R₄ and R₅ are independently
a) the side chain of a natural amino acid, or
b) optionally substituted C₁-C₄ alkyl, C₂-C₄ alkenyl, or C₂-C₄ alkynyl, or
c) —CH₂XCH₃, —CH₂CH₂XCH₃, or —CH₂XCH₂CH₃, wherein X is —O—, S, or —NR₇ wherein R₇ is hydrogen, methyl or ethyl; or
d) —CH₂Q or CH₂OQ wherein Q is as defined in relation to R₆; or
   R₄ and R₅ taken together with the carbon atom to which they are attached form an optionally substituted cycloalkyl or heterocyclic ring of 3 to 8 ring atoms, optionally fused to a second, optionally substituted, carbocyclic or heterocyclic ring.

The compounds of the invention may be regarded as amino acid derivatives of L-dopa (2-amino-3-(3,4-dihydroxyphenyl)-propanoic acid) or L-dopa-like compounds, wherein the former (to the left of the wavy line in formula (IA)) is linked to the latter (to the right of the wavy line in formula (IA)) by a peptide bond (intersected by the wavy line in formula (IA)):

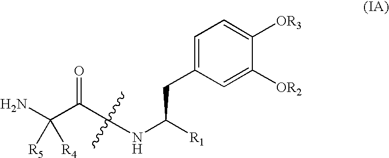

The amino acid which acylates the amino group of the L-dopa part is characterised by di-substitution on its alpha carbon atom

As used herein, the term “(Cₐ-C_{b})alkyl” wherein a and b are integers refers to a straight or branched chain alkyl radical having from a to b carbon atoms. Thus when a is 1 and b is 6, for example, the term includes methyl, ethyl, n-propyl, isopropyl, n-butyl, isobutyl, sec-butyl, t-butyl, n-pentyl and n-hexyl.

As used herein the term “(Cₐ-C_{b})alkenyl” means a straight or branched chain alkenyl moiety having from a to b carbon atoms having at least one double bond of either E or Z stereochemistry where applicable. Thus when a is 2 and b is 6, the term includes, for example, vinyl, allyl, 1- and 2-butenyl and 2-methyl-2-propenyl.

As used herein the term “C₂-C₆ alkynyl” refers to straight chain or branched chain hydrocarbon groups having from a to b carbon atoms and having in addition one triple bond. Thus when a is 2 and b is 6, the term includes, for example, ethynyl, 1-propynyl, 1- and 2-butynyl, 2-methyl-2-propynyl, 2-pentynyl, 3-pentynyl, 4-pentynyl, 2-hexynyl, 3-hexynyl, 4-hexynyl and 5-hexynyl.

As used herein the unqualified term “carbocyclic” refers to a mono-, bi- or tricyclic radical having up to 16 ring atoms, all of which are carbon, and includes aryl and cycloalkyl.

As used herein the unqualified term “cycloalkyl” refers to a monocyclic saturated carbocyclic radical having from 3-8 carbon atoms and includes, for example, cyclopropyl, cyclobutyl, cyclopentyl, cyclohexyl, cycloheptyl and cyclooctyl.

As used herein the unqualified term “aryl” refers to a mono-, bi- or tri-cyclic carbocyclic aromatic radical, and includes radicals having two monocyclic carbocyclic aromatic rings which are directly linked by a covalent bond. Illustrative of such radicals are phenyl, biphenyl and napthyl.

As used herein the unqualified term “heteroaryl” refers to a mono-, bi- or tri-cyclic aromatic radical containing one or more heteroatoms selected from S, N and O, and includes radicals having two such monocyclic rings, or one such monocyclic ring and one monocyclic aryl ring, which are directly linked by a covalent bond. Illustrative of such radicals are thienyl, benzthienyl, furyl, benzfuryl, pyrrolyl, imidazolyl, benzimidazolyl, thiazolyl, benzthiazolyl, isothiazolyl, benzisothiazolyl, pyrazolyl, oxazolyl, benzoxazolyl, isoxazolyl, benzisoxazolyl, isothiazolyl, triazolyl, benztriazolyl, thiadiazolyl, oxadiazolyl, pyridinyl, pyridazinyl, pyrimidinyl, pyrazinyl, triazinyl, indolyl and indazolyl.

As used herein the unqualified term “heterocyclyl” or “heterocyclic” includes “heteroaryl” as defined above, and in its non-aromatic meaning relates to a mono-, bi- or tri-cyclic non-aromatic radical containing one or more heteroatoms selected from S, N and O, and to groups consisting of a monocyclic non-aromatic radical containing one or more such heteroatoms which is covalently linked to another such radical or to a monocyclic carbocyclic radical. Illustrative of such radicals are pyrrolyl, furanyl, thienyl, piperidinyl, imidazolyl, oxazolyl, isoxazolyl, thiazolyl, thiadiazolyl, pyrazolyl, pyridinyl, pyrrolidinyl, pyrimidinyl, morpholinyl, piperazinyl, indolyl, morpholinyl, benzfuranyl, pyranyl, isoxazolyl, benzimidazolyl, methylenedioxyphenyl, ethylenedioxyphenyl, maleimido and succinimido groups.

Unless otherwise specified in the context in which it occurs, the term “substituted” as applied to any moiety herein means substituted with up to four compatible substituents, each of which independently may be, for example, (C₁-C₆)alkyl, (C₃-C₆) cycloalkyl, (C₁-C₆)alkoxy, hydroxy, hydroxy(C₁-C₆)alkyl, mercapto, mercapto(C₁-C₆)alkyl, (C₁-C₆)alkylthio, phenyl, monocyclic heterocyclic, benzyl, phenoxy, halo (including fluoro, bromo and chloro), trifluoromethyl, trifluoromethoxy, nitro, nitrile (—CN), oxo, —COOH, —COOR^{A}, —COR^{A}, —SO₂R^{A}, —CONH₂, —SO₂NH₂, —CONHR^{A}, —SO₂NHR^{A}, —CONR^{A}R^{B}, —SO₂NR^{A}R^{B}, —NH₂—, —NHR^{A}, —NR^{A}R^{B}, —OCONH₂, —OCONHR^{A} —OCONR^{A}R^{B}, —NHCOR^{A}, —NHCOOR^{A}, —NR^{B}COOR^{A}, —NHSO₂OR^{A}, —NR^{B}SO₂OH, —NR^{B}SO₂OR^{A}, —NHCONH₂, —NR^{A}CONH₂, —NHCONHR^{B}, —NR^{A}CONHR^{B}, —NHCONR^{A}R^{B}, or —NR^{A}CONR^{A}R^{B} wherein R^{A} and R^{B} are independently a (C₁-C₆)alkyl, (C₃-C₆) cycloalkyl, phenyl, benzyl or monocyclic heterocyclic having 5 or 6 ring atoms, or R^{A} and R^{B} when attached to the same nitrogen may, together with that nitrogen, form a 4- to 6-membered ring containing that nitrogen. An “optional substituent” may be one of the foregoing substituent groups.

As used herein the term “salt” includes base addition, acid addition and quaternary salts. Compounds of the invention which are acidic can form salts, including pharmaceutically acceptable salts, with bases such as alkali metal hydroxides, e.g. sodium and potassium hydroxides; alkaline earth metal hydroxides e.g. calcium, barium and magnesium hydroxides; with organic bases e.g. N-methyl-D-glucamine, choline tris(hydroxymethyl)amino-methane, L-arginine, L-lysine, N-ethyl piperidine, dibenzylamine and the like. Those compounds (I) which are basic can form salts, including pharmaceutically acceptable salts with inorganic acids, e.g. with hydrohalic acids such as hydrochloric or hydrobromic acids, sulphuric acid, nitric acid or phosphoric acid and the like, and with organic acids e.g. with acetic, tartaric, succinic, fumaric, maleic, malic, salicylic, citric, methanesulphonic, p-toluenesulphonic, benzoic, benzenesunfonic, glutamic, lactic, and mandelic acids and the like.

In the compounds of the invention, carbon atom to which R1 is attached is assymmetric, and the stereochemistry at that centre is as shown in formula (I). However, the compounds of the invention may contain one or more additional chiral centres, because of the presence of asymmetric carbon atoms, and they can exist as a number of diastereoisomers with R or S stereochemistry at each chiral centre. The invention includes all such diastereoisomers and mixtures thereof.

### The Group R₁

R₁ may be a carboxyl group (—COOH), a carboxyl ester group or a carboxamide group. Compounds wherein R₁ is a carboxyl group form one presently preferred subclass.

Examples of carboxyl ester groups R₁ include those of formula —COOR^{C} wherein R^{C} is a C₁-C₆ alkyl or C₂-C₆ alkenyl group. A presently preferred carboxyl ester group is the methyl ester —COOCH₃.

Examples of carboxamide groups R₁ include those of formula —CONR^{B}(Alk)ₙR^{A} wherein
Alk is an optionally substituted divalent C₁-C₆ alkylene, or C₂-C₆ alkenylene or C₂-C₆ alkynylene radical,
n is 0 or 1,
R^{B} is hydrogen or a C₁-C₆ alkyl or C₂-C₆ alkenyl group,
R^{A} is hydrogen, hydroxy or optionally substituted carbocyclic or heterocyclyl,
or R^{A} and R^{B} taken together with the nitrogen to which they are attached form an N-heterocyclic ring which may optionally contain one or more additional hetero atoms selected from O, S and N, and which may optionally be substituted on one or more ring C or N atoms.

Thus, in carboxamide groups R₁ of formula —CONR^{B}(Alk)ₙR^{A}, Alk may be optionally substituted —CH₂—, —CH₂CH₂—, —CH₂CH₂CH₂—, —CH₂CH═CH—, or —CH₂CCCH₂—; R^{B} may be hydrogen or methyl, ethyl, n- or iso-propyl, or allyl; R^{A} may be hydroxy or optionally substituted phenyl, 3,4-methylenedioxyphenyl, pyridyl, furyl, thienyl, N-piperazinyl, or N-morpholinyl; or R^{A} and R^{B} taken together with the nitrogen to which they are attached form an N-heterocyclic ring which may optionally contain one or more additional hetero atoms selected from O, S and N, and which may optionally be substituted on one or more ring C or N atoms.

A presently preferred carboxamide group R₁ is —CONH₂.

### The Groups R₂ and R₃

In the compounds of the invention R₂ and R₃ may be the same or different

In a presently preferred subclass of compounds of the invention R₂ and R₃ are each hydrogen.

In another subclass, R₂ and R₃ are independently —C(═O)R₆ or —C(═O)OR₆ wherein R₆ is methyl, ethyl, n- or isopropyl, tert-butylmethyl, or benzyl which is optionally substituted in the phenyl ring thereof.

### The Groups R₄ and R₅

In a particular subclass of compounds of the invention, at least one of R₄ and R₅ is the side chain of a natural amino acid.

In another subclass, R₄ and R₅ may independently be, for example, optionally substituted C₁-C₄ alkyl, phenyl, benzyl cyclopropyl, cyclobutyl, cyclopentyl, cyclohexyl, cyclopropylmethyl, cyclobutylmethyl, cyclopentylmethyl, cyclohexylmethyl, pyridyl, pyridylmethyl, piperidinyl, piperazinyl or morpholinyl.

Presently preferred are compounds wherein one of R₄ and R₅ is methyl, and indeed R₄ and R₅ may each be methyl.

In a further subclass of compounds of the invention, R₄ and R₅ taken together with the carbon atom to which they are attached form a C₁-C₆ cycloalkyl ring, which is optionally benz-fused, for example a cyclopropyl, cyclobutyl, cyclopentyl or cyclohexyl ring.

### Optional Substituents in any of R₁-R₅

Any optional substituents may be selected from, for example, methyl, trifluoromethyl, methoxy, trifluoromethoxy, cyclopropyl, halogen, cyano, hydroxy, mercapto, oxo, —NH₂, —NHR^{A}, or —NR^{A}R^{B} wherein R^{A} and R^{B} are independently methyl or ethyl.

### Stereochemistry

In compounds of the invention where R₄ is the side chain of a natural amino acid and R₅ is not the side chain of a natural amino acid, it is currently preferred that the stereochemical orientation of the bond between R₄ and the carbon to which it is attached should be S.

Examples of specific compounds of the invention include those of the examples herein.

### Synthetic Routes

There are multiple synthetic strategies for the synthesis of the compounds (I) with which the present invention is concerned, but all rely on chemistry known to the synthetic organic chemist. Compounds according to formula (I) can be synthesised according to procedures described in the standard literature and are well-known to the one skilled in the art. Typical literature sources are “Advanced organic chemistry”, 4^{th} Edition (Wiley), J March, “Comprehensive Organic Transformation”, 2^{nd} Edition (Wiley), R. C. Larock, “Handbook of Heterocyclic Chemistry”, 2^{nd} Edition (Pergamon), A. R. Katritzky), review articles such as found in “Synthesis”, “Acc. Chem. Res.”, “Chem. Rev”, or primary literature sources identified by standard literature searches online or from secondary sources such as “SciFinder” or “Beilstein”.

In general the compounds of the invention are accessible by well known methods of peptide synthesis whereby an acylating derivative of an amino acid (II) is reacted with the amino group of an amino acid of formula (III)

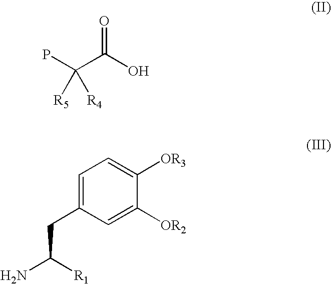

wherein P is a protected amino group and R1-R5 are as defined in relation to formula (I) above, to form the desired peptide bond between the two reactants, and the protected amino group of reactant (II) is deprotected.

### Pharmaceutical Utility

As mentioned above, many of the compounds of the invention are cleaved in the body to release L-dopa or L-dopa precursors. L-dopa itself is metabolised in the gut, the gut membrane, plasma, kidney and the liver and this significantly reduces its bioavailability and increases intersubject variability in the resulting blood levels of L-dopa. The present compounds have a different pharmacokinetic profile from L-dopa itself, due to the time over which cleavage occurs and L-dopa is released.

Hence the compounds of the present invention are useful in a method of treatment of a condition associated with impaired dopaminergic signalling in a subject, comprising administering to the subject an amount of the compound effective to reduce such impairment. The compounds are also useful in the preparation of a composition for treatment of a condition associated with impaired dopaminergic signalling. Examples of such conditions include Parkinson's disease, or Restless Legs Syndrome, as well as Tourette's syndrome, attention deficit hyperactive disorder, generation of pituitary tumours, a parkinson-plus syndrome, levodopa responsive dystonia, dyskinesia, periodic movements in sleep, dysphagia or neuroleptic malignant syndrome.

Typical examples of Parkinson's disease which can be treated with the compounds of the invention include sporadic Parkinson's disease, familial forms of Parkinson's disease and post-encephalitic Parkinsonism.

Typical examples of Parkinson-plus syndromes which can be treated with the compounds of the invention include progressive supranuclear palsy and multiple system atrophy.

Typically, the dyskinesia is L-dopa-induced dyskinesia.

The compounds of the invention may be administered in a variety of dosage forms. Thus, they can be administered orally, for example as tablets, capsules, troches, lozenges, aqueous or oily suspensions, dispersible powders or granules. The compounds can be administered in a sublingual formulation, for example a buccal formulation. The compounds of the invention may also be administered parenterally, whether subcutaneously, intravenously, intramuscularly, intrasternally, transdermally, by inhalation (e.g. intranasally) or by infusion techniques. The compounds may also be administered as suppositories. Typically, the compounds of the invention are administered orally or by inhalation (e.g. intranasally). Preferably, the compounds of the invention are administered orally. More preferably, the compounds of the invention are administered as a tablet or capsule.

The present invention further provides a pharmaceutical composition containing a compound of formula (I), or a pharmaceutically acceptable salt thereof, as defined above, and a pharmaceutically acceptable carrier.

The compounds of the invention are typically formulated for administration with a pharmaceutically acceptable carrier or diluent. For example, solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, arabic gums, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non toxic and pharmacologically inactive substances used in pharmaceutical formulations. Such pharmaceutical preparations may be manufactured in known manner, for example, by means of mixing, granulating, tableting, sugar coating, or film coating processes.

Liquid dispersions for oral administration may be syrups, emulsions and suspensions. The syrups may contain as carriers, for example, saccharose or saccharose with glycerine and/or mannitol and/or sorbitol. Suspensions and emulsions may contain as carrier, for example a natural gum, agar, sodium alginate, pectin, methylcellulose, carboxymethylcellulose, or polyvinyl alcohol. The suspension or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Since the compounds of the invention are preferably administered orally, the present invention further provides a pharmaceutical composition containing a compound of formula (I), or a pharmaceutically acceptable salt thereof, as defined above, and a pharmaceutically acceptable carrier in the form of a capsule or tablet.

Solutions for injection or infusion may contain as carrier, for example, sterile water or preferably they may be in the form of sterile, aqueous, isotonic saline solutions.

The compounds of the present invention may also be administered with a peripheral decarboxylase inhibitor. The present invention therefore provides a pharmaceutical composition containing a compound of formula (I), or a pharmaceutically acceptable salt thereof, as defined above, a peripheral decarboxylase inhibitor and a pharmaceutically acceptable carrier or diluent. Typically the peripheral decarboxylase inhibitor is carbidopa or benserazide. Preferably the peripheral decarboxylase inhibitor is carbidopa.

Also provided is a product comprising (a) a compound of the formula (I), or a pharmaceutically acceptable salt thereof, as defined above and (b) a peripheral decarboxylase inhibitor as defined above, for simultaneous separate or sequential use in the treatment of the human or animal body.

Further, said medicament is typically for co-administration with a peripheral decarboxylase inhibitor defined above.

It will be understood that the specific dose level for any particular patient will depend upon a variety of factors including the activity of the specific compound employed, the age, body weight, general health, sex, diet, time of administration, route of administration, rate of excretion, drug combination and the severity of the particular disease undergoing treatment. Optimum dose levels and frequency of dosing will be determined by clinical trial. However, it is expected that a typical dose will be in the range from about 0.001 to 50 mg per kg of body weight.

The following examples illustrate the invention:

### LC-MS Method

The system used to obtain LC-MS data comprised a Waters Alliance 2695 quaternary HPLC, Waters 996 Photo Diode Array (PDA) detector and Waters ZQ 2000 single quadrupole mass spectrometer. The ZQ can acquire data simultaneously in positive and negative electrospray ionisation modes.

### ZQ Mass Spectrometer

| | | | |
|---|---|---|---|
| Capillary | 3.3 kV/−3.0 kV | Cone | 40 V/−40 V |
| Extractor | 5 V/−5 V | Source Temp | 110° C. |
| Desolvation Temp | 400° C. | Cone Gas | 40 L/Hr |
| Desolvation Gas | 350 L/Hr | Multiplier | 500 V/−500 V |

Data were acquired in a full scan from 80 to 1000 m/z

| | |
|---|---|
| Scan duration | 0.80 s |
| Interscan delay | 0.20 s |

### HPLC

The reverse phase separation was carried out on a Zorbax XDB C8 150×4.6 mm with 5 μm silica from Agilent for both Method A and Method B.

| | |
|---|---|
| Injection Volume | 10 μL |
| UV data | 220 to 400 nm |
| Sample Temperature | 20° C. |
| Column Temperature | 30° C. |
| Flow Rate | 1.0 mL/min |
| Split to ZQ | 0.3 mL/min |

Method A (approximately pH 3.2)

| | |
|---|---|
| Solvent A | Water/10 mM NH₄HCO₂/0.1% formic acid |
| Solvent B | 95% CH₃CN/5% A/0.1% formic acid |

Method B (approximately pH 5.5)

| | |
|---|---|
| Solvent C | Water/10 mM NH₄HCO₂ |
| Solvent D | 95% CH₃CN/5% C |

### Gradient Program for Method A

| Time | A % | B % | C % | D % | Flow | Curve |
|---|---|---|---|---|---|---|
| 0.00 | 95.0 | 5.0 | 0.0 | 0.0 | 1.000 | 1 |
| 1.00 | 95.0 | 5.0 | 0.0 | 0.0 | 1.000 | 6 |
| 11.00 | 5.0 | 95.0 | 0.0 | 0.0 | 1.000 | 6 |
| 14.20 | 5.0 | 95.0 | 0.0 | 0.0 | 1.000 | 6 |
| 14.50 | 95.0 | 5.0 | 0.0 | 0.0 | 1.000 | 6 |
| 15.00 | 95.0 | 5.0 | 0.0 | 0.0 | 1.000 | 6 |

### Gradient Program for Method B

| Time | A % | B % | C % | D % | Flow | Curve |
|---|---|---|---|---|---|---|
| 0.00 | 0.0 | 0.0 | 95.0 | 5.0 | 1.000 | 1 |
| 1.00 | 0.0 | 0.0 | 95.0 | 5.0 | 1.000 | 6 |
| 11.00 | 0.0 | 0.0 | 5.0 | 95.0 | 1.000 | 6 |
| 14.20 | 0.0 | 0.0 | 5.0 | 95.0 | 1.000 | 6 |
| 14.50 | 0.0 | 0.0 | 95.0 | 5.0 | 1.000 | 6 |
| 15.00 | 0.0 | 0.0 | 95.0 | 5.0 | 1.000 | 6 |

### EXAMPLE 1

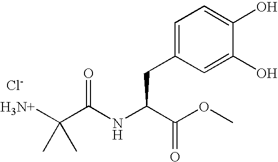

### 1-[(S)-2-(3,4-Dihydroxyphenyl)-1-methoxycarbonylethylcarbamoyl]-1-methyl-ethylammonium Chloride

### Step 1

To a solution of (S)-2-(3,4-dihydroxyphenyl)-1-methoxycarbonylethylammonium chloride (1.0 g, 4.0 mmol) in dichloromethane (20 mL) was added triethylamine (0.444 g, 4.0 mmol) DMF (5 mL), 2-tert-butoxycarbonylamino-2-methylpropionic acid (0.894, 4.40 mmol), and HOBt (0.595 g, 4.40 mmol). The mixture was stirred for 20 min. EDC (0.844 g, 4.40 mmol) was added and stirring was continued for 16 h. AcOH (0.5 mL) was added. The mixture was washed with saturated NaHCO₃ solution and saturated NaCl solution. The organic layer was dried over Na₂SO₄. The Na₂SO₄ was removed by filtration and the filtrate evaporated in vacuo. The residue was purified by column chromatography, eluting with ethyl acetate/hexane 2:1 to afford a colourless solid (1.32 g)

### Step 2

The product of Step 1 (0.60 g) was suspended in dichloromethane (5 mL) and the suspension cooled in an ice-water bath. A solution of HCl(g) in dioxane (4M, 2.5 mL) was added and the mixture stirred at 20° C. for 5 h. Ether was added and the mixture stirred for a further 30 min. The resulting precipitate was collected by filtration and dried in vacuo to afford a colourless solid (0.47 g). HPLC (Method A) retention time 4.18 min. Mass spectrum (ES+) m/z 297 (M+H).

### EXAMPLE 2

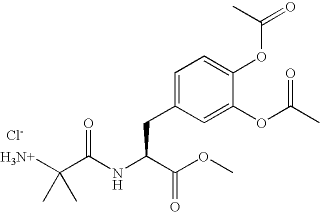

### 1-[(S)-2-(3,4-Diacetoxyphenyl)-1-methoxycarbonylethylcarbamoyl]-1-methyl-ethylammonium Chloride

The product of Example 1 Step 1 (0.52 g) was treated with acetic acid (3 mL) and heated to 80° C. It was then cooled to 40° C. and HCl (g) was bubbled through the solution. Acetyl chloride (2 mL) was added dropwise. The mixture was cooled to 20° C. and stirred for 16 h. Ether was added and the precipitate was collected by filtration to afford a colourless solid (0.47 g). HPLC (Method A) retention time 5.75 min. Mass spectrum (ES+) m/z 381 (M+H).

### EXAMPLE 3

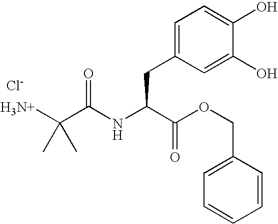

### 1-[(S)-1-Benzyloxycarbonyl-2-(3,4-dihydroxyphenyl)-ethylcarbamoyl]-1-methyl-ethylammonium Chloride

### Step 1

3,4-dihydroxy-L-phenylalanine (15.0 g, 0.076 mol) was suspended in benzyl alcohol (381 mL). This suspension was then cooled with an ice bath to 5° C. treated with thionyl chloride (76.2 mL). The resulting solution was heated to between 95° C. and 100° C. for 5 h, in a nitrogen atmosphere. The suspension was cooled to room temperature and diluted with dry ether (1.5 L) which gave a solid precipitate. The suspension was stirred at 20° C. for 16 h, filtered, and then washed with ether and dried in a vacuum oven to afford a colourless solid (7.7 g).

### Step 2

An analogous procedure to Example 1 Step 1 was followed starting from (S)-1-benzyloxycarbonyl-2-(3,4-dihydroxy-phenyl)-ethylammonium chloride (1.0 g, 3.0 mmol). The product was purified by column chromatography eluting with ethyl acetate/hexane (3:2) to give a colourless oil (1.34 g) which solidified on standing to a colourless solid (1.29 g)

### Step 3

An analogous procedure to Example 1 Step 2 was followed starting from the product of Step 2 (1.23 g) which afforded an off-white solid (1.06 g). HPLC (Method A) retention time 6.14 min. Mass spectrum (ES+) m/z 373 (M+H).

### EXAMPLE 4

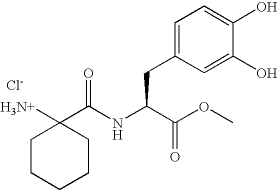

### 1-[(S)-2-(3,4-Dihydroxy-phenyl)-1-methoxycarbonyl-ethylcarbamoyl]-cyclohexyl-ammonium Chloride

### Step 1

(S)-2-(3,4-Dihydroxy-phenyl)-1-methoxycarbonylethylammonium; chloride (0.748 g, 3.01 mmol) was dissolved in dichloromethane (2 mL) and 1-tert-butoxycarbonylaminocyclohexanecarboxylic acid (0.731 g, 3.01 mmol) added followed by triethylamine (0.609 g, 6.03 mmol). HOBT (0.406 g, 3.01 mmol) was then added and the mixture stirred for min. O-(Benzotriazol-1-yl)-N,N,N′,N′-tetramethyluronium tetrafluoroborate (1.143 g, 3.01 mmol) was then added. The mixture was stirred for 36 h and then diluted with dichloromethane (20 mL) and washed with saturated NaHCO₃, 2M HCl and water and evaporated in vacuo to afford a colourless solid (1.87 g).

### Step 2

An analogous procedure to Example 1 Step 1 was followed starting from the product of Step 1 (0.179 g) which afforded an off-white solid (0.11 g). HPLC (Method A) retention time 4.78 min. Mass spectrum (ES+) m/z 337 (M+H).

### EXAMPLE 5

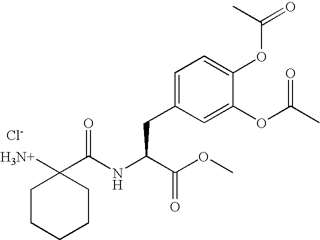

### 1-[(S)-2-(3,4-Diacetoxyphenyl)-1-methoxycarbonylethylcarbamoyl]-cyclohexyl-ammonium Chloride

An analogous procedure to Example 2 was followed starting from the product of Example 10 Step 1 (0.189 g) which afforded a colourless solid (0.11 g). HPLC (Method A) retention time 6.20 min. Mass spectrum (ES+) m/z 421 (M+H).

### EXAMPLE 6

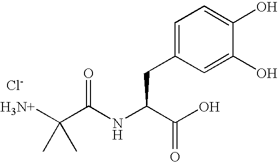

### 1-[(S)-1-Carboxy-2-(3,4-dihydroxyphenyl)-ethylcarbamoyl]-1-methylethyl-ammonium Chloride

Example 3 (0.30 g) was dissolved in water (15 mL) and treated with 5% Pd/C (0.030 g). The mixture was hydrogenated at 30 psi for 3 h. The catalyst was removed by filtration and the filtrate was evaporated in vacuo. The residue was lyophilized to afford a colourless glass (0.20 g). HPLC (Method A) retention time 2.23 min. Mass spectrum (ES+) m/z 283 (M+H).

### EXAMPLE 7

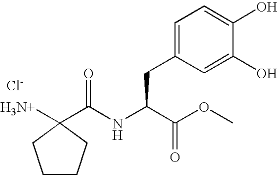

### 1-[(S)-2-(3,4-Dihydroxyphenyl)-1-methoxycarbonylethylcarbamoyl]-cyclopentyl-ammonium Chloride

### Step 1

An analogous procedure to Example 1 Step 1 was followed starting from 1-tert-butoxycarbonylaminocyclopentanecarboxylic acid (0.505 g, 2.2 mmol). The product was purified by column chromatography eluting with ethyl acetate/hexane (3:2) to afford a colourless foam (0.72 g).

### Step 2

An analogous procedure to Example 1 Step 2 was followed starting from the product of Step 1 (0.30 g) which afforded a colourless solid (0.23 g). HPLC (Method B) retention time 4.84 min. Mass spectrum (ES+) m/z 323 (M+H).

### EXAMPLE 8

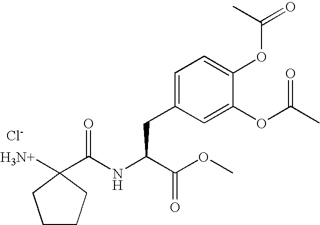

### 1-[(S)-2-(3,4-Diacetoxyphenyl)-1-methoxycarbonylethylcarbamoyl]-cyclopentyl-ammonium Chloride

An analogous procedure to Example 2 was followed starting from the product of Example 7 Step 1 (0.30 g) which afforded a colourless solid (0.28 g). HPLC (Method B) retention time 6.64 min. Mass spectrum (ES+) m/z 407 (M+H).

### EXAMPLE 9

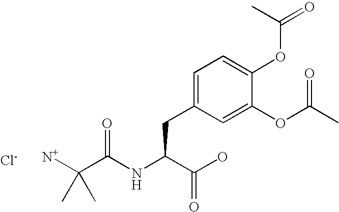

### 1-[(S)-1-Carboxy-2-(3,4-diacetoxyphenyl)-ethylcarbamoyl]-1-methylethyl-ammonium Chloride

Example 6 (0.17 g) was dissolved in AcOH (20.1 mL). HCl(g) was bubbled through the solution. The mixture was heated to 40° C. and acetyl chloride (1.4 mL) was added. The mixture was cooled to 20° C. and stirred for 16 h. Diethyl ether was added to the solution and the precipitate was collected by filtration and dried in vacuo to afford a colourless solid (0.13 g). HPLC (Method A) retention time 5.08 min. Mass spectrum (ES+) m/z 357 (M+H).

### EXAMPLE 10

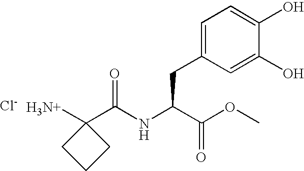

### 1-[((S)-2-(3,4-Dihydroxyphenyl)-1-methoxycarbonylethylcarbamoyl]-cyclobutyl-ammonium Chloride

### Step 1

An analogous procedure to Example 1 Step 1 was followed starting from 1-tert-butoxycarbonylaminocyclobutanecarboxylic acid (0.50 g, 2.32 mmol). The product was purified by column chromatography eluting with 5% dichloromethane/MeOH. The residue was recrystallised from hexane/ethyl acetate to afford a colourless solid (0.46 g).

### Step 2

An analogous procedure to Example 1 Step 2 was followed starting from the product of Step 1 (0.201 g) which afforded a beige solid (0.11 g). HPLC (Method A) retention time 4.33 min. Mass spectrum (ES+) m/z 307 (M−H).

### EXAMPLE 11

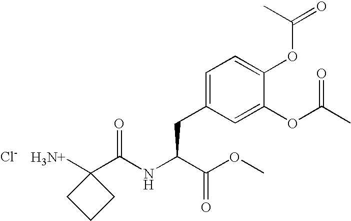

### 1-[(S)-2-(3,4-Diacetoxyphenyl)-1-methoxycarbonylethylcarbamoyl]-cyclobutyl-ammonium Chloride

An analogous procedure to Example 2 was followed starting from the product of Example 10 Step 1 (0.241 g, 0.59 mmol) which afforded a colourless solid (0.18 g). HPLC (Method A) retention time 5.84 min. Mass spectrum (ES+) m/z 393 (M+H).

### EXAMPLE 12

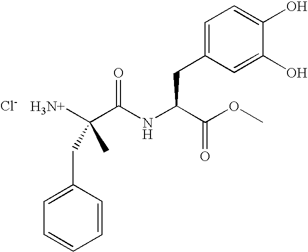

### (S)-1-[(S)-2-(3,4-Dihydroxyphenyl)-1-methoxycarbonyl-ethylcarbamoyl]-1-methyl-2-phenylethylammonium Chloride

### Step 1

An analogous procedure to Example 1 Step 1 was followed starting from (S)-2-tert-butoxycarbonylamino-2-methyl-3-phenylpropionate dicyclohexylammonium chloride (1.0 g, 2.17 mmol). The product was purified by column chromatography eluting with ethyl acetate/hexane (2:3) to afford a colourless solid (0.36 g).

### Step 2

An analogous procedure to Example 1 Step 2 was followed starting from the product of Step 1 (0.151 g) which afforded a pale yellow solid (0.11 g). HPLC (Method A) retention time 5.59 min. Mass spectrum (ES+) m/z 373 (M+H).

### EXAMPLE 13

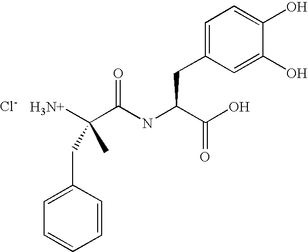

### (S)-1-[(S)-1-Carboxy-2-(3,4-dihydroxyphenyl)-ethylcarbamoyl]-1-methyl-2-phenylethylammonium Chloride

### Step 1

An analogous procedure to Example 3 Step 1 was followed starting from (S)-2-tert-butoxycarbonylamino-2-methyl-3-phenylpropionic acid (0.615 g, 2.2 mmol). The product was purified by column chromatography eluting with ethyl acetate/hexane (2:3) to afford a colourless oil (0.85 g).

### Step 2

An analogous procedure to Example 9 was followed starting from the product of Step 1 (0.75 g) to afford the a colourless solid (0.37 g).

### Step 3

An analogous procedure to Example 1 Step 2 was followed starting from the product of Step 2 (0.24 g) which afforded a colourless solid (0.20 g). HPLC (Method A) retention time 4.43 min. Mass spectrum (ES+) m/z 359 (M+H).

### EXAMPLE 14

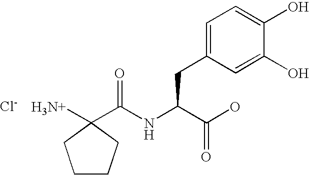

### 1-[(S)-1-Carboxy-2-(3,4-dihydroxyphenyl)-ethylcarbamoyl]-cyclopentyl-ammonium Chloride

### Step 1

A glacial acetic acid (66 ml) suspension containing 1-benzyloxycarbonyl-2-(3,4-dihydroxyphenyl)-ethylammonium chloride (3.23 g, 0.01 mol) was warmed to 110° C. HCl(g) was bubbled through the mixture for 4 min. The solution was cooled to 45° C. and acetyl chloride (10.1 ml) was added. This solution was stirred at 20° C. for 16 h and then, diluted with dry ether (150 mL). The precipitate was filtered and dried under vacuum to afford colourless crystals (3.4 g).

### Step 2

To a solution of 1-benzyloxycarbonylamino-cyclopentanecarboxylic acid (1 mmol) in dichloromethane was added (S)-1-benzyloxycarbonyl-2-(3,4-diacetoxy-phenyl)-ethyl-ammonium; chloride (1 mmol) at 0° C. followed by PyBOP (1 mmol) and DIPEA (2 mmol). The reaction was allowed to warm to 20° C. and stirred at 20° C. for 12 h. The reaction mixture was washed with 1 M HCl followed by satd. aq. NaHCO₃. The organic layer was dried and concentrated to obtain the crude product, which was purified by flash chromatography eluting with ethyl acetate:hexane (stepwise gradient from 100% hexane to 100% ethyl acetate).

### Step 3

The product of Step 3 (1 mmol) was dissolved in THF (20 mL) and 1M HCl (5 mL) was added. This was followed by addition of 10% Pd/C (50% wet, 600 mg). The resulting suspension was hydrogenated at 1 atm H₂ for 24 h. The catalyst was removed by filtration and the filtrate was concentrated in vacuo. The residue was purified by flash chromatography eluting with 10% MeOH/dichloromethane. The product was dissolved in MeCN: H₂O (1:1) and lyophilized to afford a yellow solid (0.104 g). HPLC (Method A) retention time 3.08 min. Mass spectrum (ES+) m/z 309 (M+H).

### EXAMPLE 15

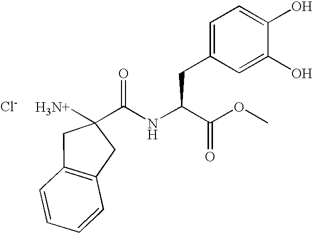

### 2-[(S)-2-(3,4-Dihydroxyphenyl)-1-methoxycarbonylethylcarbamoyl]-indan-2-yl-ammonium Chloride

### Step 1

An analogous procedure to Example 1 Step 1 was followed starting from 2-tert-butoxycarbonylamino-indan-2-carboxylic (0.25 g, 0.9 mmol). The product was purified by column chromatography eluting with ethyl acetate/hexane (3:2) to afford a colourless solid (0.28 g).

### Step 2

An analogous procedure to Example 1 Step 2 was followed starting from the product of Step 1 (0.13 g, 0.276 mmolg) which afforded a pale yellow solid (0.11 g). HPLC (Method A) retention time 5.87 min. Mass spectrum (ES+) m/z 371 (M+H).

### EXAMPLE 16

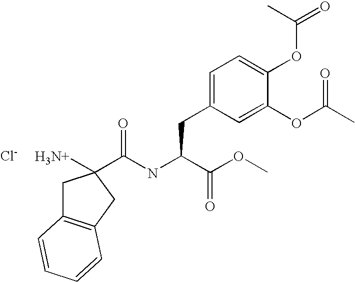

### 2-[(S)-2-(3,4-Diacetoxyphenyl)-1-methoxycarbonylethylcarbamoyl]-indan-2-yl-ammonium Chloride

An analogous procedure to Example 2 was followed starting from the product of Example 10 Step 1 (0.13 g, 0.276 mmol) which afforded a colourless solid (0.28 g). HPLC (Method A) retention time 6.71 min. Mass spectrum (ES+) m/z 455 (M+H).

### EXAMPLE 17

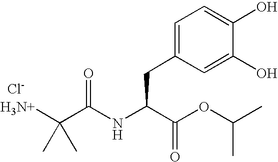

### 1-[(S)-2-(3,4-Dihydroxyphenyl)-1-isopropoxycarbonylethylcarbamoyl]-1-methyl-ethylammonium Chloride

### Step 1

3,4-dihydroxy-L-phenylalanine (3.0 g) was suspended in 2-propanol (20 mL). HCl(g) was bubbled through the suspension for 10 min. The mixture was heated to reflux for 4 h, cooled to 20° C. and ether added. The precipitate was collected by filtration and dried in vacuo to afford a colourless solid (3.1 g).

### Step 2

An analogous procedure to Example 1 Step 1 was followed starting from the product of Step 1 (0.551 g, 2.0 mmol). The product was purified by column chromatography eluting with ethyl acetate/hexane (3:2) to give a colourless solid (0.69 g).

### Step 3

An analogous procedure to Example 1 Step 2 was followed starting from the product of Step 2 (0.37 g) which afforded a colourless solid (0.31 g). HPLC (Method A) retention time 5.23 min. Mass spectrum (ES+) m/z 325 (M+H).

### EXAMPLE 18

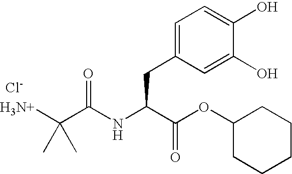

### (S)-1-Cyclohexyloxycarbonyl-2-(3,4-dihydroxyphenyl)-ethylammonium Chloride

### Step 1

An analogous procedure to Example 17 Step 1 was followed starting from 3,4-dihydroxy-L-phenylalanine (3.11 g) and cyclohexanol (20 mL) to afford a colourless solid (3.1 g)

### Step 2

An analogous procedure to Example 1 Step 1 was followed starting from the product of Step 1 (0.755 g, 2.4 mmol). The product was purified by column chromatography eluting with ethyl acetate/hexane (1:1) to give a colourless solid (0.68 g).

### Step 3

An analogous procedure to Example 1 Step 2 was followed starting from the product of Step 2 (0.37 g) which afforded a colourless solid (0.30 g). HPLC (Method A) retention time 6.29 min. Mass spectrum (ES+) m/z 365 (M+H).

### EXAMPLE 19

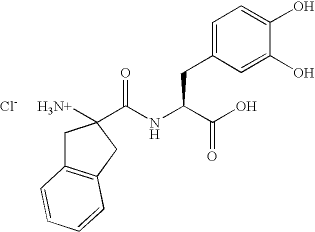

### 2-[(S)-1-Carboxy-2-(3,4-dihydroxyphenyl)-ethylcarbamoyl]-indan-2-yl-ammonium Chloride

### Step 1

To a solution of 2-tert-butoxycarbonylaminoindan-2-carboxylic acid (1 mmol) in dichloromethane was added at 0° C. the product of Example 3 Step 1 (1 mmol) followed by PyBOP (1 mmol) and DIPEA (2 mmol). The reaction was allowed to warm to 20° C. and stirred at 20° C. for 12 h. The reaction mixture was washed with 1 M HCl followed by satd. aq. NaHCO₃. The organic layer was dried and concentrated to obtain the crude product, which was purified by flash chromatography eluting with ethyl acetate:hexane (stepwise gradient from 100% hexane to 100% ethyl acetate).

### Step 2

The product of Step 1 (1 mmol) was dissolved in THF (20 mL) and 10% Pd/C (50% wet, 600 mg) was added. The resulting suspension was hydrogenated at 1 atm H₂ for 24 h. The catalyst was removed by filtration and the filtrate was concentrated in vacuo. The residue was purified by flash chromatography eluting with 5% MeOH/dichloromethane. The residue was dissolved in dioxane (10 mL), cooled to 15° C. and saturated with HCl (g). The reaction mixture was stirred for 12 h after which the solvent was removed in vacuo. The residue was purified by flash chromatography on silica gel eluting with 10% MeOH/dichloromethane to afford a green solid (0.070 g). HPLC (Method A) retention time 4.57 min. Mass spectrum (ES+) m/z 357 (M+H).

### EXAMPLE 20

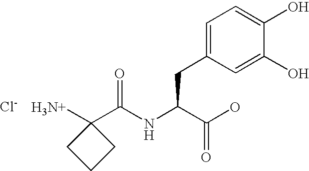

### 1-[(S)-1-Carboxy-2-(3,4-dihydroxyphenyl)-ethylcarbamoyl]-cyclobutyl-ammonium Chloride

### Step 1

To a solution of 1-benzyloxycarbonylamino-cyclobutanecarboxylic acid (1 mmol) in dichloromethane at 0° C. was added the product of Example 3 Step 1 (1 mmol) followed by PyBOP (1 mmol) and DIPEA (2 mmol). The reaction was allowed to warm to 20° C. and stirred at 20° C. for 12 h. The reaction mixture was washed with 1 M HCl followed by satd. aq. NaHCO₃. The organic layer was dried and concentrated in vacuo. The residue was purified by flash chromatography eluting with ethyl acetate:hexane (stepwise gradient from 100% hexane to 100% ethyl acetate).

### Step 2

The product of Step 1 (1 mmol) was dissolved in THF (20 mL) and 10% Pd/C (50% wet, 600 mg) was added. The resulting suspension was hydrogenated at 1 atm H₂ for 24 h. The catalyst was removed by filtration and the filtrate was concentrated in vacuo. The residue was purified by flash chromatography eluting with 10% MeOH/dichloromethane. The product was dissolved in MeCN: H₂O (1:1) and lyophilized to afford a brown solid (0.075 g). HPLC (Method A) retention time 1.49 min. Mass spectrum (ES+) m/z 295 (M+H).

### EXAMPLE 21

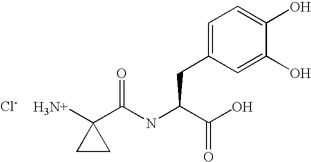

### 1-[(S)-1-Carboxy-2-(3,4-dihydroxyphenyl)-ethylcarbamoyl]-cyclopropyl Ammonium Chloride

An analogous procedure to Example 14 was followed starting from 1-benzyloxycarbonylaminocyclopropanecarboxylic acid (1 mmol) which afforded a beige solid (0.105 g). HPLC (Method A) retention time 2.17 min. Mass spectrum (ES+) m/z 281 (M+H).

### EXAMPLE 22

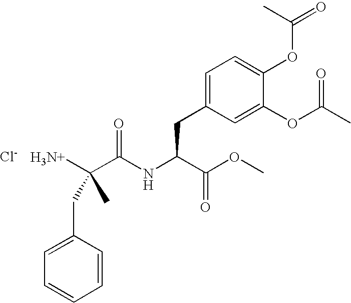

### (S)-1-[(S)-2-(3,4-Diacetoxyphenyl)-1-methoxycarbonylethylcarbamoyl]-1-methyl-2-phenylethylammonium Chloride

An analogous procedure to Example 2 was followed starting from the product of Example 12 Step 2 (0.21 g, 0.44 mmol) which afforded a beige solid (0.10 g).). HPLC (Method A) retention time 6.76 min. Mass spectrum (ES+) m/z 457 (M+H).

### EXAMPLE 23

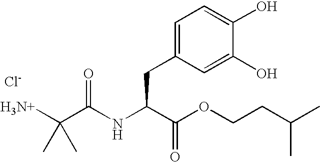

### 1-[(S)-2-(3,4-Dihydroxyphenyl)-1-(3-methylbutoxycarbonyl)-ethylcarbamoyl]-1-methylethylammonium Chloride

### Step 1

An analogous procedure to Example 17 Step 1 was followed starting from 3,4-dihydroxy-L-phenylalanine (3.1 g) and 3-methyl-1-butanol (20 mL) which afforded a colourless solid (4.1 g).

### Step 2

An analogous procedure to Example 1 Step 1 was followed starting from the product of Step 1 (0.607 g, 2.0 mmol). The product was purified by column chromatography eluting with ethyl acetate/hexane (1:1) which afforded a colourless solid (0.72 g).

### Step 3

An analogous procedure to Example 1 Step 2 was followed starting from the product of Step 2 (0.50 g) which afforded a colourless solid (0.35 g). HPLC (Method A) retention time 6.32 min. Mass spectrum (ES+) m/z 353 (M+H).

### EXAMPLE 24

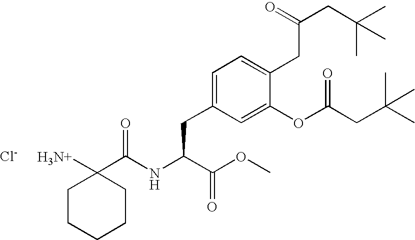

### 1-{(S)-2-[3,4-Bis-(3,3-dimethylbutyryloxy)-phenyl]-1-methoxycarbonyl-ethylcarbamoyl}-cyclohexylammonium Chloride

### Step 1

The product of Example 4 Step 1 was dissolved in dichloromethane. tert-Butylacetyl chloride (0.202 g, 1.5 mmol) was added followed by triethylamine (0.47 mL, 14.89 mmol) and DMAP (0.007 g, 0.06 mmol). The mixture was heated to 70° C. for 7 h. The mixture was cooled to 20° C. and partitioned between dichloromethane and water. The organics were washed with 1M HCl, saturated NaHCO₃ and saturated NaCl and dried over Na₂SO₄. The Na₂SO₄ was removed by filtration and the filtrate evaporated in vacuo to give an off-white solid which was purified by flash column chromatography eluting with hexane/ethyl acetate (5:1 to 3:1) which afforded a colourless solid (0.31 g, 81%).

### Step 2

An analogous procedure to Example 1 Step 2 was followed starting from the product of Step 2 (0.16 g, 0.25 mmol) which afforded a colourless solid (0.125 g). HPLC (Method A) retention time 9.56 min. Mass spectrum (ES+) m/z 533 (M+H).

TABLE
Summary of LCMS data

| Example | LCMS | retention | ES+ m/z |
|---|---|---|---|
| no. | method | time | (M + H) |
| 1 | A | 4.18 | 297 |
| 2 | A | 5.75 | 381 |
| 3 | A | 6.14 | 373 |
| 4 | A | 4.78 | 337 |
| 5 | A | 6.2 | 421 |
| 6 | A | 2.23 | 283 |
| 7 | B | 4.84 | 323 |
| 8 | B | 6.64 | 407 |
| 9 | A | 5.08 | 357 |
| 10 | A | 4.33 | 307* |
| 11 | A | 5.84 | 393 |
| 12 | A | 5.59 | 373 |
| 13 | A | 4.43 | 359 |
| 14 | A | 3.08 | 309 |
| 15 | A | 5.87 | 371 |
| 16 | A | 6.71 | 455 |
| 17 | A | 5.23 | 325 |
| 18 | A | 6.29 | 365 |
| 19 | A | 4.57 | 357 |
| 20 | A | 1.49 | 295 |
| 21 | A | 2.17 | 281 |
| 22 | A | 6.76 | 457 |
| 23 | A | 6.32 | 353 |
| 24 | A | 9.56 | 533 |

*ES− m/z (M − H). No molecular ion was observed in ES+ mode.

### Biological Results

Compounds of the examples above were tested in the following animal model of dopamine deficiency behaviour:

### Assessment of Activity in 6-OHDA-Lesioned Rats

| | |
|---|---|
| Animals | Male Wistar rats, 250 g, Harlan Ltd. |
| Housing | Animals were housed in groups of 4 on a 12-h light-dark cycle |
| | with an environment of 50% humidity and temperature of 21 ± |
| | 2° C. in accordance with Animals (Scientific Procedures) Act |
| | 1996 Home Office regulations. Rats had access to food and |
| | water ad libitum. |
| Licence | All animals used in this study were treated in accordance with |
| | the UK 1986 Animals (Scientific Procedures Act). |

### Procedure

Surgery Male Wistar rats were treated with desipramine (25 mg/kg ip, 30 minutes prior to 6-OHDA) to protect noradrenergic terminals. Rats were then anaesthetized in an induction chamber using isofluorane (1-2% in 95% O₂, 5% CO₂ carrier gas), placed in a Kopf stereotaxic frame and anaesthesia maintained with 0.5-1.0% isofluorane. An incision was made in the scalp and a 0.8-mm-diameter hole made in the skull at coordinates AP: −0.26 mm L: +2.0 mm (all co-ordinated measured from bregma). The neurotoxin 6-hydroxydopamine (6-OHDA) (8 μg free base in 4 μL of 0.9% saline containing 0.05% ascorbic acid) was injected into the left median forebrain bundle at a constant rate over 4 min (1 μl/min) using a 10-μL Hamilton syringe lowered to −8 mm below the dura. The needle remained in place for a further 4 min before being removed, and the wound cleaned and sutured. Flunixin hydrochloride (2.5 mg/kg, Dunlop's Veterinary Supplies, Dumfries, UK) was administered for pain relief and a rehydration treatment of 5% glucose in 0.9% saline (up to 5 ml ip) was given prior to recovery from the anaesthetic.

### Behavioural Assessment

### Confirmation of the Lesion

At least 2 weeks following surgery, animals were examined for rotational behaviour (see below) in response to the administration of apomorphine hydrochloride (0.5 mg/kg s.c. in 0.9% saline containing 0.05% ascorbic acid) to evaluate the extent of the lesion. Only those rats exhibiting >6 turns/min at peak activity were used in future studies.

### Assessment of the Induction of Rotational Activity by Test Compounds

At least 1 week after apomorphine administration, rats (n=4-8 per treatment) were tested for rotational activity with either a test drug or L-DOPA. These were administered either via the intraperitoneal (ip) route or orally by gavage (po). Animals were treated with benserazide (10 mg/kg) and placed in rotometers (Med Associates) for up to 30 min to measure basal activity. They were then treated with test compound or L-DOPA (63.4 μmole/kg ip or po). Rotation behaviour was assessed for up to 4 hours after test drug/L-DOPA administration. Animals were typically treated with a series of compounds for comparative purposes. Each treatment was administered at least 1 day apart.

### Data Analysis

The number of rotations measured per 10 minutes over the 4 hour period was determined. Animals were considered active if they turned >10 turns per 10 minutes. From this data the following parameters were measured:
   A Total activity (AUC activity, where AUC=area under the locomotor-activity/time curve)
   B Peak activity
   C Duration of activity
Values are quoted as % of L-DOPA induced effects.

### Results:

| | | | peak activity | duration of |
|---|---|---|---|---|
| | | AUC activity | (as % of L- | activity (as % |
| | route | (as % of L- | DOPA | of L-DOPA |
| example | of | DOPA AUC | maximal | duration of |
| number | admin. | activity) | activity) | activity) |
| 1 | i.p. | 69 | 77 | 63 |
| 2 | p.o. | 55 | 69 | 73 |
| 3 | p.o. | 46 | 71 | 38 |
| 4 | p.o. | 88 | 107 | 84 |
| 5 | p.o. | 42 | 55 | 42 |
| 6 | p.o. | 65 | 80 | 57 |
| 7 | p.o. | 78 | 105 | 97 |
| 8 | p.o. | 39 | 61 | 44 |
| 9 | p.o. | 42 | 66 | 45 |
| 10 | p.o. | 76 | 62 | 73 |
| 11 | p.o. | 75 | 93 | 67 |
| 12 | p.o. | 32 | 42 | 52 |
| 13 | p.o. | 26 | 37 | 40 |
| 14 | p.o. | 82 | 119 | 62 |
| 15 | p.o. | 41 | 69 | 31 |
| 16 | p.o. | 41 | 64 | 21 |
| 17 | p.o. | 116 | 149 | 72 |
| 18 | p.o. | 105 | 82 | 70 |
| 19 | | not tested | not tested | not tested |
| 20 | p.o. | 37 | 59 | 37 |
| 21 | p.o. | 53 | 58 | 76 |
| 22 | p.o. | 31 | 64 | 19 |
| 23 | p.o. | 79 | 59 | 49 |
| 24 | p.o. | 61 | 62 | 36 |

1. A compound of formula (I) or a salt, hydrate or solvate thereof: 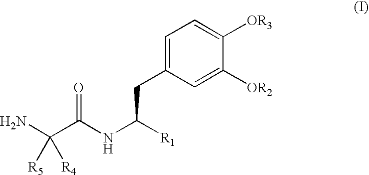
wherein:
R₁ is a carboxyl, carboxyl ester, or carboxamide group;
R₂ and R₃ are independently hydrogen, or a group —C(═O)R₆ or —C(═O)OR₆ wherein R₆ is C₁-C₆ alkyl, or a group —CH₂Q wherein Q is an optionally substituted monocyclic carbocyclic or heterocyclyl ring of 3 to 6 ring atoms;
R₄ and R₅ are independently
(a) the side chain of a natural amino acid, or
(b) optionally substituted C₁-C₄ alkyl, C₂-C₄ alkenyl, or C₂-C₄ alkynyl, or
(c) —CH₂XCH₃, —CH₂CH₂XCH₃, or —CH₂XCH₂CH₃, wherein X is —O—, S, or —NR₇ wherein R₇ is hydrogen, methyl or ethyl; or
(d) —CH₂Q or CH₂OQ wherein Q is as defined in relation to R₆; or
R₄ and R₅ taken together with the carbon atom to which they are attached form an optionally substituted cycloalkyl or heterocyclic ring of 3 to 8 ring atoms, optionally fused to a second, optionally substituted, carbocyclic or heterocyclic ring

2. A compound as claimed in claim 1 wherein R₁ is a carboxyl group.

3. A compound as claimed in claim 1 wherein R₁ is a carboxyl ester group of formula —COOR^{C} wherein R^{C} is a C₁-C₆ alkyl or C₂-C₆ alkenyl group.

4. A compound as claimed in claim 3 wherein R^{C} is methyl or.

5. A compound as claimed in claim 1 wherein R₁ is —CONH₂.

6. A compound as claimed in claim 1 wherein R₂ and R₃ are each hydrogen.

7. A compound as claimed in claim 1 wherein R₂ and R₃ are independently —C(═O)R₆ or —C(═O)OR₆ wherein R₆ is methyl, ethyl, n- or isopropyl, tert-butylmethyl, or benzyl which is optionally substituted in the phenyl ring thereof.

8. A compound as claimed in claim 8 wherein R₂ and R₃ are the same.

9. A compound as claimed in claim 1 wherein at least one of R₄ and R₅ is the side chain of a natural amino acid.

10. A compound as claimed in claim 1 wherein R₄ and R₅ are independently optionally substituted C1-C4 alkyl, phenyl, benzyl cyclopropyl, cyclobutyl, cyclopentyl, cyclohexyl, cyclopropylmethyl, cyclobutylmethyl, cyclopentylmethyl, cyclohexylmethyl, pyridyl, pyridylmethyl, piperidinyl, piperazinyl or morpholinyl.

11. A compound as claimed in claim 9 wherein one of R₄ and R₅ is methyl.

12. A compound as claimed in claim 1 wherein R₄ and R₅ are each methyl.

13. A compound as claimed in any of claim 1 wherein R₄ and R₅ taken together with the carbon atom to which they are attached form a C₁-C₆ cycloalkyl ring, which is optionally benz-fused.

14. A compound as claimed in claim 1 wherein R₄ and R₅ taken together with the carbon atom to which they are attached form a cyclopropyl, cyclobutyl, cyclopentyl or cyclohexyl ring.

15. A compound as claimed in claim 1 wherein any optional substituents are selected from methyl, trifluoromethyl, methoxy, trifluoromethoxy, cyclopropyl, halogen, cyano, hydroxy, mercapto, oxo, —NH₂, —NHR^{A}, or —NR^{A}R^{B} wherein R^{A} and R^{B} are independently methyl or ethyl.

16. A compound as claimed in claim 1 wherein R₄ is the side chain of a natural amino acid and R₅ is not the side chain of a natural amino acid, and the stereochemical orientation of the bond between R₄ and the carbon to which it is attached is S.

17. A pharmaceutical composition comprising a compound as claimed in claim 1 together with a pharmaceutically acceptable carrier.

18. (canceled)

19. A method of treatment of a condition associated with impaired dopaminergic signalling in a subject, comprising administrating to the subject an amount of a compound as claimed in claim 1 effective to reduce such impairment of dopaminergic signalling.

20. The method as claimed in claim 19, wherein the condition is Parkinson's disease, or Restless Legs Syndrome

21. The method as claimed in claim 19, wherein the condition is Tourette's syndrome, attention deficit hyperactive disorder, generation of pituitary tumours, a parkinson-plus syndrome, levodopa responsive dystonia, dyskinesia, periodic movements in sleep, dysphagia or neuroleptic malignant syndrome.
